(19) United States
(12) Patent Application Publication
(10) Pub. No.: US 2010/0172830 A1
(43) Pub. Date: Jul. 8, 2010

(54) Extraembryonic Tissue cells and method of use thereof

(75) Inventor: Mohammad A. Heidaran, Chatham, NJ (US)

Correspondence Address: MOHAMMAD A. HEIDARAN, 88 ORMONT RD., CHATHAM, NJ, 07928, US

(73) Assignee: Cellx Inc., Chatham, NJ (US)

(21) Appl. No.: 12/028,756

(22) Filed: Feb. 8, 2008

Related U.S. Application Data
(60) Provisional application No. 60/908,704, filed on Mar. 29, 2007.

(51) Int. Cl. A61K 51/00; C12N 5/00; C12N 5/02; A61K 35/12; A61K 49/00

(52) U.S. Cl. 424/1.17; 435/325; 435/378; 424/93.7; 424/9.3

(57) ABSTRACT

The present invention provides Pre-term Placenta Extra-embryonic Tissue Cell (PPETC) populations, and methods of culturing, proliferating and expanding the same. The invention also provides methods of using such cells for therapeutic and diagnostic applications. The present invention provides a method for isolation of extra-embryonic cell population will which are to be obtained preferably from premature discarded placenta by a process of non-enzymatic digestion or mechanical disruption. The isolated cells are cultured in semi-solid glycosaminoglycan-based three-dimensional milieu supplemented with autologous umbilical cord-derived serum. The isolated cells which are expanded in semi-solid media will be formulated for intravenous injections to patients having end stage cystic fibrosis. A preferred method of formulating cells for intravenous injection and optimal cell number are described herein. The PPETC may also used to diagnose diseased organs and elicit a robust immune response to stem cell markers commonly found in certain type of cancer stem cells.

### RELATED U.S. PATENT DOCUMENTS

Application Number

Filing Date

Pat. No.

Disclosure Document

May 24, 1999

457,045

Provisional Patent application

Mar. 29, 2007

60/908,704

### FIELD OF THE INVENTION

The present invention provides Pre-term Placenta Extra-embryonic Tissue Cell (PPETC) populations, and methods of culturing, proliferating and expanding the same. The invention also provides methods of using such cells for therapeutic and diagnostic applications

### BACKGROUND OF THE INVENTION

Human progenitor cells are capable of generating a variety of mature human cell lineages. Evidence exists that demonstrates that stem cells can be employed to repopulate many, if not all, tissues and restore physiologic and anatomic functionality.

Many different types of mammalian stem and progenitor cells have been characterized. See, e.g., Caplan et al., U.S. Pat. No. 5,486,359 (human mesenchymal stem cells); Boyse et al., U.S. Pat. No. 5,004,681 (fetal and neonatal hematopoietic stem and progenitor cells).

The placenta is a particularly attractive source of stem cells. Because mammalian placentas are plentiful and are normally discarded as medical waste, they represent a unique source of medically-useful cells. The present invention provides such isolated placenta-derived cells and preferred use of such cells.

### SUMMARY OF THE INVENTION

The present invention provides a method for isolation of Extra-embryonic cell population will which are to be obtained preferably from premature discarded placenta by a process of non-enzymatic digestion or mechanical. The isolated cells are cultured in semi-solid glycosaminoglycan-based three-dimensional milieu supplemented with autologous umbilical cord-derived serum. The isolated cells which are expanded in semi-solid media will be formulated for intravenous injections to patients having end stage cystic fibrosis. A preferred method of formulating cells for intravenous injection and optimal cell number are described herein. The PPETC may also used to diagnose diseased organs and elicit a robust immune response to stem cell markers commonly found in certain type of cancer stem cells.

Isolation and Propagation of Placental Stem Cells

Premature placentas are obtained from donors with informed consent. The immature placenta have been shown to be a superior source of highly multipotential. The placenta is stored according to method described in the accompanied invention disclosure of May 1999. Briefly placenta is exsanguished and placed in bag containing 100 ml of UW solution. The preferred method of shipment will be at 4° C. To generate PPETC using sterile aseptic techniques a small portion of the placenta tissue approximately about 10 to 18 mm in diameter was obtained using trephine. The tissue cubes are washed extensively for two to four hours at room temperature using three changes of saline supplemented with antibacterial agents. This step which will remove any residual blood is also designed to select for stress resistant cell population. Unlike other methods described in the literature where the maternal and fetal tissues are separated care will not be taken to avoid the maternal fetal interface. It is hypothesized that cell type of fetal and maternal origin are more effective at creating micro-chimerism in the appropriate recipients and are more effective in mediating tissue repair regeneration or modification of certain diseases. Following the stress/or wash step the tissue cube is dissected sagittally. Half of the blocked is fixed in four percent formalin the other half of cubes are pulled and minced to smaller pieces with dimension less than 1 mm×1 mm×1 mm. Minced tissues are then placed in a container which is connected to a supply of cell dis-aggregating solution. The preferred dis-aggregating solution is EDTA. The solution is passed through the tissue compartment at medium to high flow rate. Because the tissue chamber is equipped with a screen that allows single cell suspension to exit the chamber while trapping the larger fragments, this process will yield a single suspension cell type which can be collected and use for further analysis. In another embodiment the cell isolation step involves mechanical disruption followed by enzymatic digestion as described below: Mince tissue will be digested with 300 units per ml collagenase (Lakewood N.J.) and dipase II (two units per ML) for one hour at 37°. The enzymes are resuspended in Dulbecco's modify Eagle medium (DMEM) containing glucose glutamine. The cell suspension is collected and the enzymatic activity of collagenase and dipase is terminated using heat inactivated serum 2-5%.

For non-enzymatic method of cell isolation the minced tissue block approximately 1 mm3 in volume are washed in PBS followed by PBS plus 1 mM EDTA for 5 minutes. The tissue blocks are then rinsed and cultured in tissue culture plastic plates using the preferred cell expansion media (DMEM plus 10% matched human umbilical cord serum). To establish PPETC The placental stem cells are resuspended at concentration of 1×105 per ml in 1.5% sodium alginate solution and dripped into a solution of 3.5% CaCl2 solution. The approximate jelling time of 10 minutes is the most optimal. This process result in encapsulation of placental cells in alginate micro-beads which is supplemented with the preferred DMEM basal media supplemented with collagen type I (10 ug/ml), human basic Fibroblast Growth Factor (bFGF)(10 ng/ml) and laminin (1 ug/ml).

Following long term culture of placental derived cells encapsulated in alginate beads approximately 1-2 weeks. The cells are recovered from the alginate micro-beads using 55 mM sodium citrate. PPETC recovered from micro-beads culture are frozen in 90% serum and 10% DMSO.

Cryopreservation of PPETC may also be performed as described below: The placenta-derived stem cells (PPETC) can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 10% (v/v). PPETC are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use.

Characterization of PPETC: PPETC are characterized by the presence of number of progenitor cell markers including but not limited to the presence of Cystic fibrosis transmembrane receptor (CFTR) and Low density lipoprotein receptor 6 (LRP6). (Biochemical and Biophysical Research Communications Volume 248, Issue 3, 30 Jul. 1998, Pages 879-888).

Flow Cytometric and histochemical analysis of isolated and propagated cells: Single-cell suspension (100,000) were fixed with 5% formalin for 5 minutes. The fixation reaction was stopped by adding phosphate buffered saline (PBS)/1% BSA. Whenever antibodies that recognize an intracellular portion of CFTR protein (C terminus or R domain) were used, cells were permeabilized and blocked with PBS/0.1% saponin/5% dried milk for 24 hours at 4 C. Otherwise cells were with PBS/5% dried milk for 24 hours at 4 C. Cells were then incubated with primary monoclonal antibody; mouse anti-CFTR C terminous Genzyme (Cambridge Mass.), 2503-01 mouse monoclonal anti-CFTR extracellular doamin affinity Bioreagents (Golden, Colo.), MA1-935 for 1 hour in PBS/0.1% saponin/5% dried milk or PBS/5% dried milk and incubated with appropriate secondary antibody 9 rabbit anti-mouse IgM-FITC, Biosource (Carmarillo, Calif.) for additional 1 hour. The isotype control antibody used were purified mouse IgG1 or mouse IgM (Pharmingen San Diego).

Flow cytometric analysis show significantly higher expression of CFTR as compared to CFTR expression in A549 which shows 1-5% cell surface expression with Anti-CFTR antibody tested. For RT PCR analysis total RNA was extracted from cell collected following the confluence. Primers specific to human CFTR and LRP5/6 were designed and used to perform semi quantitative RT PCR analysis. Total RNA was isolated using RNAWIZ (Ambion). RT-PCR analysis was performed and the signal was normalized to similar level of human actin mRNA.

Tissue/Organ Homing property of PPETC: The isolated cells have characteristics which is suitable for efficient homing of the transplanted cells into the lung tissue. In cystic fibrosis patients this unique property of the cells will improve the localization of the exogenous cells into lung tissue (see example VI)

Preferred use of PPETC to treat Cystic Fibrosis: Cystic fibrosis (CF) is a chronic, progressive, and frequently fatal genetic (inherited) disease of the body's mucus glands. CF primarily affects the respiratory and digestive systems in children and young adults. Sweat glands and the reproductive system also are usually involved. On average, individuals with CF have a lifespan of about 30 years. CF is caused by mutations in the CFTR (cystic fibrosis transmembrane conductance regulator) gene. Heterozygous carriers (those who have inherited only one copy of the altered gene) are asymptomatic. Two altered genes must be present for CF to appear. This means that if both parents are CF carriers, their offspring would only express CF symptoms if they had inherited one defective copy of the CFTR gene from each parent. According to data collected by the Cystic Fibrosis Foundation, about 30,000 Americans, 3000 Canadians, and 20,000 Europeans have CF. The disease occurs mostly in whites whose ancestors came from northern Europe, although it affects all races and ethnic groups. Accordingly, it is less common in African Americans, Native Americans, and Asian Americans. About 2500 babies are born with CF each year in the United States. Also, about 1 in every 20 Americans is an unaffected carrier of an abnormal CF gene. These 12 million people usually are unaware that they are carriers. CF does not follow the same pattern in all patients but affects different people in different ways and to varying degrees. The basic problem, however, is the same-an abnormality in the glands that produce or secrete sweat and mucus. Sweat cools the body; mucus lubricates the respiratory, digestive, and reproductive systems and prevents tissues from drying out, protecting them from infection.

Diagnosis and Genetic Testing

Sweat test is the most common test for CF. It measures the amount of salt (sodium chloride) in the sweat. Immunoreactive Trypsinogen Test (IRT) is used for newborns who do not produce enough sweat for the sweat test. In the IRT test, blood drawn 2 to 3 days after birth is analyzed for a specific protein called trypsinogen. Positive IRT tests must be confirmed by sweat and other tests. Other tests that can assist in the diagnosis of CF include chest X rays, lung function tests, sputum (phlegm) cultures, and stool examinations to help identify typical digestive abnormalities. Molecular Genetic Testing involves carrier screening and direct DNA analysis. Current tests, however, cannot detect all of the more than 900 gene mutations, and so the tests are only 80% to 85% accurate. CF once was always fatal in childhood. Better treatment methods developed over the past 20 years have increased the average lifespan of CF patients. At present, neither gene therapy nor any other kind of treatment exists for the basic causes of CF, although several drug-based approaches are being investigated. In the meantime, doctors can only ease the symptoms of CF or slow the progress of the disease so the patient's quality of life is improved. This is achieved by antibiotic therapy combined with treatments to clear the thick mucus from the lungs. The therapy is tailored to the needs of each patient. For patients whose disease is very advanced, lung transplantation may be an option (Tait, Jonathan F., et al. (Updated 12 Apr. 2001). Cystic Fibrosis. In: GeneReviews at GeneTests-GeneClinics: Medical Genetics Information Resource [database online]. Copyright, University of Washington, Seattle. 1997-2001. Available Accessed 20 May 2002).

The preferred protocol for the treatment of cystic fibrosis patients is outlined below: The most optimal target population will be infants or fetuses which are diagnosed with the autosomal recessive disease using genetic testing or other functional tests as described in the background section. The conventional therapy today focuses on the treatment of the overall symptom associated with this disease and involves a number of drugs which are designed to clear the lung by inducing the coughing mechanism in the patients. Because this disease is attributed by the loss of CFTR gene the aim of clinical study would be to evaluate in utero or systemic infusion of partially matched cells isolated from healthy sibling placenta could restore the function of the lung to the acceptable range. The cellular characteristic of extra-embryonic cells as determined by CFTR positivity and expression of key cell surface receptors for efficient homing and engraftment to the lung are expected to alter the disease progression significantly.

The optimal cell type to be used for intravenous injection would be cells isolated based on method described herein leading to the production of highly characterized CFTR+ cells which co-express one of the following markers CXCR4, HGF or MMP1. The optimal cell dosage to be used will be approximately 1 million to 10 million cells per kg. The cell dose is typically provided to physician as cryopreserved sample. Briefly the cryopreserved sample formulated in 10% DMSO, FBS, BSA will be thawed carefully at 37° C. The sample is tested for viability and after dilution with equal volume of vehicle will be infused to the patient at rate of 100,000 cell per minute. The patient will receive antihistamine to reduce any allergic reaction and will be monitored continuously during the infusion process. Patients will also receive Pulmozyme using a nebulizer and is now widely used in older children and adults with cystic fibrosis. In adults and older children, studies have shown that daily use of Pulmozyme improves lung function and decreases the number of lung infections requiring hospital treatment. Pulmozyme has been approved by the Food and Drug Administration for use in children over 5 years old and adults with cystic fibrosis.

This study will compare cell infusion with or without Pulmozyme to a placebo. During the study infants and young children with cystic fibrosis will be treated with cell transplantation augmented with Pulmozyme for 6 months and placebo for 6 months. The study medicines will be inhaled at home once a day from a nebulizer for a period of one year. Half of the children will be treated with Pulmozyme for the first 6 months of the study and half will receive the placebo. The efficacy will be measured using infant lung function tests and by doing a special 3-D x-ray of the child's chest (a high resolution CT or HRCT) at the beginning of the study, at 6 months and at 12 month after starting study. The study will not change the regular clinical care.

Primary Outcome Measures: (1) Chest CT (HRCT Score) (2) Infant Pulmonary Function Tests (FEV0.5, FEF25-75) Secondary Outcome Measures (1) Hospital days; (2)Antibiotic treatment days. Inclusion Criteria: (1) Age <30 months (2) Diagnosis of CF based on clinical features consistent with CF as well as 1 of the 2 following criteria: a) two sweat chlorides >60 mEq/L (by quantitative pilocarpine iontophoresis), b) genotype with 2 identifiable mutations consistent with CF. (3) Informed consent by parent or legal guardian

Exclusion Criteria: (1) Previous treatment with Pulmozyme (2) Hospitalization or treatment with IV antibiotics with 14 days of initial study visit (3)Acute intercurrent respiratory infection, defined as any of the following symptoms within the preceding 48 hours: 1) fever >38 degrees C., 3a) new onset of coryza or other upper respiratory symptoms, 3b) increase in cough, wheezing, or respiratory rate History of adverse reaction to sedation (4) Oxyhemoglobin saturation <90% on room air (5) Severe upper airway obstruction as determined by site PI (severe laryngomalacia, markedly enlarged tonsils, significant snoring, diagnosed obstructive sleep apnea) (6)Hemodynamically significant congenital heart disease or diagnosed arrhythmias (7) History of hemoptysis (8) History of previous pulmonary air leak (pneumothorax) (9) Diagnosed seizure disorder necessitating current anticonvulsive therapy. A history of febrile seizures is not an exclusion criterion. (10) Use of Investigational drug(s) within 60 days or 5 half-lives of enrollment in this study. (11) Known allergy to Chinese Hamster Ovary-derived biological products or any component of the placebo or active drug formulations.

Method of intrauterine transplantation of CFTR+ cells

To treat patients in utero a suspension of 10 million cells in 7 ml of media will be infused the umbilical vein by technique similar to that used for intravesular intrauterine transfusion. At birth the chimerism is detected by monitoring the percentage of donor cells using antibody against HLA class I a antigens (Touraine J L et al., In-utero transplantation of stem cells in bare lymphocyte syndrome Lancet 1382, 1989).

Method of inducing chimerism using placental CFTR+ cells for creating efficient chimerism in the recipients the patients are subjected to reduce intensity myeloablation therapy including the use of alemtuzumab, fludarabine and melphalan. The cell population consisting of both fetal and maternal cells may be used to establish chimerism in the related recipients. In a preferred embodiment the recipient is minimally myeloablated and transfused with a mix population of cells derived from both maternal and fetal compartment of the placenta. In another embodiment several independently derived cell population isolated from well defined placenta source are combined to create a super chimera dose for transplantation. In clinical setting only one of the transplanted chimeric cell population will achieve long term engraftment. The ability of using cell population derived from independent cell source will increases the likelihood of achieving long term engraftment and chimerism in the host.

PPETC Derived Cancer Vaccine:

In another embodiment the placenta extra-embryonic cells (PPETC) are used as vaccine to stimulate the immune system of cancer patients. It has been shown that placenta extra-embryonic cells share markers with cancer stem cells. These surface molecules include receptors for Wnt signaling pathways, receptor tyrosine kinases such as Vascular Endothelial Growth Factor Receptor (VEGFR1 and 2) and G-coupled receptors. A conventional methodology to stimulate patient immune system involves the use of whole tumor or RNA derived from tumors which are expressed by antigen presenting cells isolated from the patients. According to the invention described we intent to use PPETC to induce a robust immunological response to markers normally expressed in cancer stem cells. The ability to induce the immunological to cancer stem cells will be a more efficient methodology to block proliferation of late stage tumors. Conventional therapies rely heavily on the expression of tumor specific antigens isolated from whole tumor biopsy. Because cancer stem cells constitute a very small portion of tumor cells population it is virtually impossible to elicit a robust immunological response to the cancer stem cells. The invention described herein makes it possible to generate an allogeneic off-the shelf cellular product which can be used to immunized patients with cancer. The use of off the shelf cellular vaccine will elicit immune response to the small population of tumor responsible for uncontrolled of cancerous cells. Because PPETC share cell surface characteristics similar to cancer stem cells the development of such immuno-therapeutics is not dependent on the availability of patient specific cancer stem cells. PPETC share common ABC transporter gene expression profile as several human lung cancer cell line. There are 50 known ABC transporters present in humans, which are classified into seven families by the Human Genome Organization. These include ABCA, ABCB, ABCC and ABCG2. These finding suggest that PPETC may be a useful stimulator of patient specific immunity against specific cancers such as lung cancer (Cancer Research 67, 4827-4833, May 15, 2007).

PPETC in Vivo Cancer Diagnostics:

Yet in another embodiment we propose to use the autologous or allogeneic PPETC labeled by a variety of dyes detectable by MRI to detect diseased organ or cancer before the disease become diagnosable using conventional strategies. The preferred method of using PPETC is for diagnosis of early stages of lung disease. These include CF, lung fibrosis and lung cancer. The lung is the primary site for efficient homing of PPETC. In the first 24 hours in healthy adults PPETC will be cleared from normally functioning lung. In patients with early stages of fibrosis or cancer the PPETC will remain in lung for additional 24 hours. As such the detection of labeled PEC in the lung after 48 hours will be the diagnostic marker for early stages of severe lung disease.

The PPETCs express high level of a receptor well known to the field as a chemo-attractant to SDF-1 a major cytokine secreted by diseased organs or cancerous cells. The PPETC in another embodiment is treated with agents that upregulate CXCR4 expression, labeled using specific dye as described and intravenously injected. The location of labeled PEC may be detected by non-invasive imaging technology such as MRI when sufficient concentration of such cells localized to an injury site or site that contain excessive SDF-1 expression as detected in many solid tumors. The ability to develop patient specific PPETC will enable the development of highly sensitive methodology to detect diseased organ or cancerous tissue. Because this process can detect as many as few aberrant cells the process is considered to be non invasive and more sensitive than any conventional immuno-diagnostics.

In another embodiment this application also teaches a new method by which stem cells can be used for diagnostic application to detect abnormal cells expressing high levels of SDF-1, HGF-1 or MMP1 According to method described herein PPET will be culture expanded in a cocktail containing several factors inducing the up-regulation of CXCR4 or HGF receptor. The placental stem cells exhibiting upregulation of at least one of the following receptors (CXCR4, HGF receptor c-Met and PDGFRs alpha and beta) will be labeled with 111In oxine and used to detect diseased or cancerous cells using SPECT/CT.

The following protocol may be used to label PPETC to detect low number of circulating tumor cell line in Nude animals: To produce nude mice bearing tumor cell line was selected as it is commonly used for evaluation of new cancer treatments and can produce highly aggressive solid tumors when injected subcutaneously into mice. Cells were grown in T75 flasks in Weymouth media containing 15% fetal calf serum and sodium bicarbonate under standard tissue culture conditions (37° C.) with humidified atmosphere of 5% CO2 with balance air). On the day of the experiment, the media from one flask was removed and replaced with 1 ml-0.25% trypsin. Detached cells were then suspended in 8 ml media and centrifuged at 300×g for 5 min. The cell pellet was resuspended in 8 ml media and counted with the assistance of a hemocytometer. The volume of cell suspension was adjusted to achieve a cell density of 1 million cells/ml.

Quantum dot labeling of cells: Labeling of PPETC cells with quantum dots was performed with a Qtracker™ cell labeling kit (Quantum Dot Corp., Hayward, Calif.). A 40 nM stock solution of QD was prepared according to the manufacturer's protocol.

A 500 μl aliquot of QD stock was added to 10 ml of cell suspension and incubated for 1 h at 37° C. The cell suspension was centrifuged at 300×g for 5 min and the supernatant was replaced with 10 ml media. Optical images were collected using predefined filter settings: excitation used a filter optimized for the organic dye DsRed (˜500).

Methods of Obtaining Stromal PPETC (SPPETC):

Pre-term placenta is recovered from a patient after informed consent and after a complete medical history of the patient is taken and is associated with the placenta.

Prior to recovery of placenta-derived stem cells, the umbilical cord blood and placental blood are removed. Typically a needle or cannula is used, with the aid of gravity, to exsanguinate the placenta. Typically, a placenta is transported from the delivery or birthing room to another location, e.g., a laboratory, for recovery of cord blood and collection of PPETC/SPPETC cells. The placenta is preferably transported in a sterile, thermally insulated bag which contains 100 ml of preservation media such as UW Solution. The organ is transported from hospital to processing center at 4 Co. The device for transportation of the placenta was described in appendix Disclosure document # 457045dated May 18, 1999.

Physical Disruption and Enzymatic Digestion of Placental Tissue: SPPETC are collected from a mammalian placenta by physical disruption, e.g., enzymatic digestion, of the organ. A portion of placenta is removed and minced. Any method of physical disruption can be used, provided that the method of disruption leaves a plurality, more preferably a majority, and more preferably at least 60% of the cells in said organ viable, as determined by trypan blue exclusion assay.

Following physical disruption minced tissues is further disrupted by the addition of a chelator, e.g., ethylene glycol bis(2-aminoethyl ether)-N,N,N′N′-tetraacetic acid (EGTA) or ethylenediaminetetraacetic acid (EDTA). Disrupted tissue is cultured in 10% human derived serum in DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), PDGF-A, bFGF, EGF, IGF-1, and penicillin/streptomycin; DMEM-LG (low glucose).

During cell culture process the non adherent cell population is continuously obtained. To remove non adherent cells the tissue culture plastic containing minced tissue is washed following media change initially every day for 3 days and there on every 3 days. The non adherent cells collected are pooled and cryopreserved. The non adherent cells are commonly referred to as PPETC-NA or SPPETC. In some cases cell recovery is facilitated by addition of digestion enzymes include, e.g., 50-200 U/mL for collagenase I and collagenase IV, 1-10 U/mL for dispase, and 10-100 U/mL for elastase.

After 3 weeks of culturing minced tissue individual colonies emerge using tissue culture plastic. typically several 10-100 individual colonies are pooled by a process commonly referred to as trypsinization. placental cells obtained by perfusion or digestion can, for example, be further, or initially, isolated by differential trypsinization using, e.g., a solution of 0.05% trypsin with 0.2% EDTA (sigma, St. Louis Mo.). Differential trypsinization is possible because placenta-derived stem cells typically detach from plastic surfaces within about five minutes whereas other adherent populations typically require more than 20-30 minutes incubation. The detached placenta-derived stem cells can be harvested following trypsinization and trypsin neutralization, using, e.g., trypsin neutralizing solution (tns, cambrex).

In one embodiment of isolation of adherent cells (SPPETC) or non-adherent cells (PPETC-NA) are propagated in 3D alginate beads as described in Examples.

To facilitate the propagation of CFTR+ cells the alginate beads are modified with extracellular matrix type I/VI collagen and laminin and the cell encapsulated in alginate beads are cultured in 10% human umbilical cord derived serum in DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), PDGF-A, bFGF, EGF, IGF-1, and penicillin/streptomycin; DMEM-LG (low glucose).

In one embodiment of isolation of adherent cells (SPPETC-Ad) or an-adherent cells (PPETC-NA) aliquots of, for example, about 5-10×106 cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO2). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

Assays for Characterization and Enrichment of PPETC/SPPETC

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CFTR, one can determine, using the techniques above, whether a cell comprises a detectable amount of CFTR; if so, the cell is CFTR+ Likewise, if a cell produces enough CFTR and LRP5/6 RNA to be detectable by RT-PCR.

PPETC may be characterized using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning. In another embodiment, the invention provides a method of identifying a compound that modulates growth, cell cycle arrest and/or differentiation of PPETC, comprising contacting said stem cells with said compound under conditions that allow growth, differentiation or cell cycle arrest.

Example I shows the approximate anatomical region of the placental tissue. The process involves 4 steps: Step 1: Placenta procurement and removal of residual blood; Step 2: Isolation of extraembryonic tissue block; Step 3: Wash step; Step 4: Non-Enzymatic and mechanical cell isolation; step 5 involves culturing of minced tissue to generate discrete colonies which are trypsinized, washed and cryopreserved.

Example II: Method of isolating and propagating of unique cell population in placenta: The placental stem cells are resuspended at concentration of 1×105 per ml in 1.5% sodium alginate solution and dripped into a solution of 3.5% CaCl2 solution. The approximate jelling time of 10 minutes is the most optimal. This process result in encapsulation of placental stem cells in alginate micro-beads which is supplemented with the following extracellular matrix proteins Collagen Type I, VI, human basic Fibroblast Growth Factor and laminin and 10% human umbilical cord derived serum.

Example III: RT-PCR analysis of identifier genes expressed in propagated cells: For RT PCR analysis total RNA was extracted from cell collected following the confluence. Primers specific to human CFTR and LRP5/6 were designed and used to perform semi quantitative RT PCR analysis. Total RNA was isolated using RNAWIZ (Ambion). RT-PCR analysis was performed and the signal was normalized to similar level of human actin mRNA. Expression of CFTR gene and LRP5 genes were quantiated and comapred to that shown by bone marrow derived mesenchymal cells. These results will be confirmed by immunohistochemical analysis and flow cytometry.

Example IV: Methods of establishing chimerism in myeloablated patients: A diverse bank of PPETC is created that covers the major HLA loci. Based on HLA profile of patients, several PPETC is pooled formulated and infused in the patients.

Example Va: Method of enhancing patient specific anti cancer immunity: Comarisom of Expression levels of of several genes in PPETC as compared to Human human lung cancer cell 1 (H460, H23, HTB-58, A549, H441, and H2170). Total RNA isolated from several lung cancer cell line as well as PPETC were subjected to RT-PCR analysis using primer specific to human ATP transporter gene ABCG2. The expression level to be norrmalized to similar level of actin mRNA. Example Vb: Method of using allogeneic PPETC as a cancer vaccine: PPETC 100 million will be formulated in appropriate vehicle/adjuvant and systemically or locally infused to the cancer patients. The cell infusion may be performed repeatedly for additional 2-3 times to elicit a robust anti- tumor response. The preferred patient population are diagnosed with late stage lung cancer.

Example VI: Preferred Method of generating labeled PPETC for diagnostic application. Nude mice were injected with highly aggressive tumor cell line with preferred site of hyperplasia in the lung. PPETC Cells were grown in T75 flasks in Weymouth media containing 15% fetal calf serum and sodium bicarbonate under standard tissue culture conditions (37° C.) with humidified atmosphere of 5% CO2 with balance air). On the day of the experiment, the media from one flask was removed and replaced with 1 ml-0.25% trypsin. Detached cells were then suspended in 8 ml media and centrifuged at 300×g for 5 min. The cell pellet was resuspended in 8 ml media and counted with the assistance of a hemocytometer. The volume of cell suspension was adjusted to achieve a cell density of 1×106 cells/ml. 111In oxine labeling may be performed by suspension of PPETC with 111In oxine 500 micro Ci for 20 minutes at room temp. labeled cells are washed and used for in vivo studies. Quantum dot labeling of cells: Labeling of PEC cells with quantum dots was performed with a Qtracker™ cell labeling kit (Quantum Dot Corp., Hayward, Calif.). A 40 nM stock solution of QD was prepared according to the manufacturer's protocol. A 500 μl aliquot of QD stock was added to 10 ml of cell suspension and incubated for 1 h at 37° C. The cell suspension was centrifuged at 300×g for 5 min and the supernatant was replaced with 10 ml media. Optical images were collected using predefined filter settings: excitation used a filter optimized for the organic dye DsRed (˜500).

### REFERENCES

Search Result: WWW.USPTO.GOV

7,255879 Title: Post-partum mammalian placenta, its use and placenta stem cells therefrom

7,141,549 Title: Protein and nucleic acid encoding the same

7,122,345 Title: Nucleic acid encoding a NOVX13 polypeptide

6,964,849 Title: Proteins and nucleic acids encoding same

Search Result WWW.NCBI.NLM.NIH.GOV:

1: Novel therapies for the treatment of cystic fibrosis: new developments in gene and stem cell therapy. Clin Chest Med. 2007 June; 28(2):361-79. Review.

2: Airway epithelium directed gene therapy for cystic fibrosis. Med Chem. 2006 September; 2(5):499-503. Review.

3: Gene therapy progress and prospects: cystic fibrosis. Gene Ther. 2006 July; 13(14):1061-7. Review. PMID: 16819538 [PubMed—indexed for MEDLINE]

4: Gene and cell therapy for cystic fibrosis. Paediatr Respir Rev. 2006;7 Suppl 1:S163-5. Epub 2006 Jun. 6. Review. PMID: 16798550 [PubMed—indexed for MEDLINE]

5: Stem cells and cystic fibrosis. J Cyst Fibros. 2006 August, 5(3):141-3. Epub 2006 Mar. 6. Review. PMID: 16574502 [PubMed—indexed for MEDLINE]

6: Transbronchial biopsies provide longitudinal evidence for epithelial chimerism in children following sex mismatched lung transplantation. Thorax. 2005 January; 60(1):60-2. PMID: 15618585 [PubMed—indexed for MEDLINE]

7: Embryonic stem cells generate airway epithelial tissue. Am J Respir Cell Mol Biol. 2005 February; 32 (2):87-92. Epub 2004 Dec. 2. PMID: 15576671 [PubMed—indexed for MEDLINE]

8: The potential for stem cell therapy in cystic fibrosis. J R Soc Med. 2004;97 Suppl 44:52-6. Review. No abstract available. PMID: 15239295 [PubMed—indexed for MEDLINE]

### OTHER REFERENCES

Pesce et al, Stem Cells 2001; 19:271-8.

Abbott, Hematol Oncol 2003; 21:115-130.

Wobus et al, Physiol Rev 2005; 85:635-78.

Huss, Stem Cells 2000; 18:1-9. Abkowitz, 2002, Can human hematopoietic stem cells become skin, gut, or liver cells? N Engl J Med. 346(10):770-2.

Cole et al., 1985, EBV-Hydradoma techique and its application to human lung cancer. In Monoclonal Antibodies and Cancer Therapy, Alan R. Liss, Inc., 77-96.

Cote et al., 1983, Generation of human monoclonal antibodies reactive with cellular antigens. Proc Natl Acad Sci U S A. 80(7):2026-30.

Damjanov et al., 1993, Retinoic acid-induced differentiation of the developmentally pluripotent human germ cell tumor-derived cell line, NCCIT. Lab Invest. 68(2):220-32. DeLoia et al, 1998, Effects of methotrexate on trophoblast proliferation and local immune responses. Hum Reprod. 13(4):1063-9.

Douay et al, 1995. Characterization of late and early hematopoietic progenitor/stem cell sensitivity to mafosfamide. Bone Marrow Transplant. 15(5):769-75.

Dushnik-Levinson et al. 1995, Embryogenesis in vitro: study of differentiation of embryonic stem cells. Biol Neonate. 67(2):77-83.

Genbacev et al. 1995, Maternal smoking inhibits early human cytotrophoblast differentiation. Reprod Toxicol. 9(3):245-55.

Hatzopoulos et al. 1998, Isolation and characterization of endothelial progenitor cells from mouse embryos. Development. 125(8):1457-68.

Himori, et al . 1984, Chemotherapeutic susceptibility of human bone marrow progenitor cells and human myelogenous leukemia cells (HL-60) in co-culture: preliminary report. Int J Cell Cloning. 2(4):254-62.

Hirashima et al. 1999, Maturation of embryonic stem cells into endothelial cells in an in vitro model of vasculogenesis. Blood. 93(4):1253-63.

Keown et al., 1990, Methods for introducing DNA into mammalian cells. Methods Enzymol. 185:527-37.

Korbling et al., 2002, Hepatocytes and epithelial cells of donor origin in recipients of peripheral-blood stem cells. N Engl J Med. 346(10):738-46.

Kozbor et al., 1983, The production of monoclonal antibodies from human lymphocytes. Immunology Today 4, 72-79. Lowy et al. 1980, Isolation of transforming DNA: cloning the hamster aprt gene. Cell. 22(3):817-23.

Melchner, et al., 1985, Human placental conditioned medium reverses apparent commitment to differentiation of human promyelocytic leukemia cells (HL60). Blood. 66(6):1469-72.

Mulligan and Berg, 1981 Selection for animal cells that express the Escherichia coli gene coding for xanthine-guanine phosphoribosyltransferase Proc Natl Acad Sci U S A. 78(4):2072-6.

Nadkarni et al. 1984, Effect of retinoic acid on bone-marrow committed stem cells (CFU-c) from chronic myeloid leukemia patients. Tumori. 70(6):503-5. Cited by other.

O'Hare et al. 1981, Transformation of mouse fibroblasts to methotrexate resistance by a recombinant plasmid expressing a prokaryotic dihydrofolate reductase. Proc Natl Acad Sci U S A. 78(3):1527-31.

Ray et al., 1997, CYP26, a novel mammalian cytochrome P450, is induced by retinoic acid and defines a new family. J Biol Chem. Jul. 25, 1997; 272(30):18702-8.

Reyes et al. 2002, Origin of endothelial progenitors in human postnatal bone marrow. J Clin Invest. 109 (3):337-46.

Santerre et al., 1984, Expression of prokaryotic genes for hygromycin B and G418 resistance as dominant-selection markers in mouse L cells. Gene. 30(1-3):147-56. Slager 1993, Transforming growth factor-beta in the early mouse embryo: implications for the regulation of muscle formation and implantation. Dev Genet. 14(3):212-24.

Smithies et al. 1985, Insertion of DNA sequences into the human chromosomal beta-globin locus by homologous recombination. Nature. 317(6034):230-4.

Szybalska and Szybalska, 1962, Genetics of human cell lines IV: DNA-mediated heritable transformation of a biochemical trait. PNAS 48: 2026-2034. Cited by other.

Thomas and Capecchi, 1987, Site-directed mutagenesis by gene targeting in mouse embryo-derived stem cells. Cell. 51(3):503-12.

Tremblay et al., 2001, Diethylstilbestrol regulates trophoblast stem cell differentiation as a ligand of orphan nuclear receptor ERR beta. Genes Dev. 15(7):833-8.

Uchimura et al. 1998, Human N-acetylglucosamine-6-O-sulfotransferase involved in the biosynthesis of 6-sulfo sialyl Lewis X: molecular cloning, chromosomal mapping, and expression in various organs and tumor cells. J Biochem (Tokyo). 124(3):670-8. Viacord, 2001, Umblicical cord blood can save lives (Informational brochure), Boston: ViaCell CENTR-BRO R1 October 2001.

Wigler et al. 1997, Transfer of purified herpes virus thymidine kinase gene to cultured mouse cells. Cell. 11(1):223-32.

Yan et al., 2001, Retinoic acid promotes differentiation of trophoblast stem cells to a giant cell fate. Dev. Biol. 235(2):422-32.

Contractor et al., A comparison of the effects of different perfusion regimes on the structure of the isolated human placental lobule. Cell Tissue Res. 237:609-617 (1984). Moore et al., “A simple perfusion technique for isolation of maternal intervillous blood mononuclear cells from human placentae”, J. Immunol. Methods 209:93-104 (1997).

Shamblott et al., “Derivation of pluripotent stem cells from cultured human primordial germ cells”, Proc. Natl. Acad. Sci. USA 95:13726-13731 (1998). Erratum in: Proc. Natl. Acad. Sci. U S A 96:1162 (1999).

Thompson et al., “Embryonic stem cell lines derived from human blastocysts”, Science 282:1145-1147 (1998); Erratum in: Science 282:1827 (1998).

Turner et al., “A modified harvest technique for cord blood hematopoietic stem cells”, Bone Marrow Transplant. 10:89-91 (1992).

Wang et al., “Enhanced recovery of hematopoietic progenitor and stem cells from cultivated postpartum human placenta”, Blood 98(11/1):183a, abstract No. 769 (2001). Ye et al., “Recovery of placental-derived adherent cells with mesenchymal stem cell characteristics”, Blood 98(11/1):147b, abstract No. 4260 (2001).

Addison et al., “Metabolism of Prednisolone by the Isolated Perfused Human Placental Lobule”, J. Ster. Biochem. Mol. Biol., vol. 39 No. 1, pp. 83-90 (1991). cited by other.

Barry, 1994, “Where do all the placentas go?” Canadian Journal of Infection Control 9(1):8-10.

Belvedere et al., 2000, “Increased blood volume and CD34(+)CD38(−) progenitor cell recovery using a novel umbilical cord blood collection system,” Stem Cells 18(4):245-251.

Elchalal et al., 2000, “Postpartum unbilical cord blood collection for transplantation: a comparison of three methods,” Am. J. of Obstetrics & Gyn. 182(1 Pt 1):227-232. Caplan, Clin Plast Surg 21(3):429-435 (1994).

CD34, Medline Mesh Database, 2004.

Cord Blood Stem Cell, Mesh Term Database, 2003.

Bersinger et al., Reproduct Fertil Dev 4:585-588 (1992).

Ma et al., Tissue Engineering 5:91-102 (1999).

MacLaren et al., J Comp Pathol 106:279-297 (1992).

Madri et al., J Cell Biol 97:153-165 (1983).

Minguell et al., Exp Biol Med 226:507-520 (2001).

Muhlemann et al., Placenta 16:367-373 (1995).

Myllynen, dissertation, University of Oulu, 2003.

Oppenheim et al., Theriogenology 55:1567-1581 (2001).

Papaioannou et al., Stem Cells Handbook:19-31 (2004).

Placenta, Mesh Pubmed, 2003.

Stromberg, et al., Methods in Cell Biol 21:227-252 (1980).

Tissue Culture: Merriam-Webster's Online Dictionary, 2004.

Totipotent Stem Cells, Stem Cells Information Center Online, 2004.

Totipotent Stem Cells, Medline, Mesh Database, 2004.

Van Bekkum, Verh Dtsch Ges Patol 74:19-24 (1990).

Expansion of human cord blood CD34=CD38+ cells in ex vivo culture during retroviral transduction without a corresponding increase in SCID repopulating cell (SRC) frequency: dissociation of SRC phenotype and function(Blood, vol. 95, No. 1, p. 102-110, January 2000) C. Dorrell, 2000.

Ex Vivo Expansion of Hematopoietic Precursors, Progenitors, and Stem Cells: The Next Generation of Cellular Therapeutics (Blood, vol. 87, No. 8, p. 3082-3088 (April 1996)) Stephen G. Emerson, 1996.

Ex Vivo Expansion of Hematopoietic Stem and Progenitor Cells: Are We There Yet? (The Journal of Hematotherapy, vol. 8, p. 93-102 (1999)) Srour, et al.

What is claimed:

1. An isolated and propagated cell population that is CFTR+ from pre-term discarded placenta

2. The isolated stem cell of claim 1, wherein said stem cell is LRP5/6+ and CFTR+

3. The isolated stem cell of claim 1, wherein said propagated in three-dimensional beads and supplemented with media optimized for extra-embryonic cell proliferation

4. The isolated stem cell of claim 1, wherein said cell facilitates formation of one or more extra-embryoid-like bodies when cultured in alginate beads and supplemented with serum obtained from matched umbilical cord blood.

5. Method of isolating cell of claim 1, wherein said cultured in alginate beads or similar materials and supplemented with 2-10% serum obtained from matched umbilical cord blood.

6. Method of isolating cell of claim 1, wherein said cultured in alginate beads or similar materials and supplemented with 2-10% serum obtained from matched umbilical cord blood, bFGF (10 ng/ml); PDGF-A (30 ng/ml); Type VI collagen (10 ug/ml) and IGF-I (30 ng/ml).

7. Method of formulating PPETC for localized implantation whereas PPETC combined with Umbilical-cord-derived plasma rich platelet preparation for local delivery or delivered in appropriate three-dimensional culture environment such as modified extracellular matrices.

8. A method of treating lung fibrosis associated with lung disease or cystic fibrosis using systemic infusion of PPETC to patients.

9. A method of using PPETC to treat cystic fibrosis patients by infusing CFTR+ positive cells systemically

10. A method of treating cystic fibrosis patent pre-birth by infusing PPETC cells into the fetus umbilicus vein directly intra-uterinely.

11. A method of diagnosing cystic fibrosis and treating fetus prebirth intra-uterinely using PPETC derived from sibling placenta.

12. A method of diagnosis for early stages of lung disease whereby culture expanded cells are labeled infused and their distribution to the lung is assessed by SPECT/CT or MRI

13. A method of diagnosis for early stages of lung disease whereby culture expanded cells are labeled by 111In oxine or quantum dot infused and their distribution to the lung is assessed by SPECT/CT or MRI 24, 48 hours, 68 hours and 124 hours post infusion

14. A method of inducing immune response to tumor stem cells in vivo by administrating irradiated PPETC population formulated in an appropriate adjuvant.

15. A method of stimulating cancer patient immune response by systemic or direct infusion of minimally or highly mismatched PPETC.

16. A method of claim 14 whereby cancer patients are diagnosed with solid tumors such as pancreatic, breast, lung, ovary, prostate and stomach.

17. A method of claim 14 whereby cancer patients are diagnosed with lung cancer such as small cell carcinoma.

18. A method of claim 14 whereby cancer patients are diagnosed and infused intra-tumorely with allogeneic PPETC

19. A method of inducing chimerism in patients using a combination of several independently derived PPETC.

20. A method of inducing chimerism in pediatric patients using a combination of several independently derived PPETC delivered intra-uterinely by direct injection of PPETC to the umbilicus vein.

21. A method of using PPETC expressing ABCG2, CFTR and or LRP5 as drug target.

22. A method by which PPETC is used to screen for antagonists or modulators of at least one of the following genes ABCG2, CFTR or LRP5.
